(19) United States
(12) Patent Application Publication
(10) Pub. No.: US 2025/0377364 A1
(43) Pub. Date: Dec. 11, 2025

(54) REAGENT, METHOD, AND DEVICE FOR DETECTING ALBUMIN

(71) Applicant: NEC Corporation, Tokyo (JP)

(72) Inventor: Katsumi MAEDA, Tokyo (JP)

(73) Assignee: NEC Corporation, Tokyo (JP)

(21) Appl. No.: 19/230,354

(22) Filed: Jun. 6, 2025

(30) Foreign Application Priority Data
Jun. 10, 2024 (JP) 2024-093705
Jan. 30, 2025 (JP) 2025-013609
Jun. 4, 2025 (JP) 2025-093316

(51) Int. Cl. G01N 33/68; C07F 5/00; G01N 21/64; G01N 21/77

(52) U.S. Cl. CPC G01N 33/6872; C07F 5/003; G01N 21/64; G01N 21/77; G01N 2021/7759; G01N 2333/76

(57) ABSTRACT

One aspect of the present disclosure relates to a reagent for detecting albumin, the reagent containing a terbium complex represented by a predetermined formula. In addition, one aspect of the present disclosure relates to a method for detecting albumin in a sample using the terbium complex represented by a predetermined formula. Another aspect of the present disclosure relates to a test paper for detecting albumin including a reagent containing a terbium complex represented by a predetermined formula.

This application is based upon and claims the benefit of priority from Japanese patent application No. 2024-093705, filed on Jun. 10, 2024, Japanese patent application No. 2025-013609, filed on Jan. 30, 2025, and Japanese patent application No. 2025-093316, filed on Jun. 4, 2025, the disclosures of which are incorporated herein in their entirety by reference.

### TECHNICAL FIELD

The present disclosure relates to a reagent for detecting albumin, a method for detecting albumin in a sample, a terbium complex used for detecting albumin, a device for detecting albumin, a test paper for detecting albumin, and the like.

### BACKGROUND ART

Albumin is a protein that has a molecular weight of about 66,000 and accounts for 50 to 65% of the proteins present in serum. Albumin has an action of maintaining the osmotic pressure of blood, and further has a function of binding to and transporting hormones, fatty acids, drug molecules, metal ions, and the like. Albumin is known to be associated with diseases such as kidney disease, cardiovascular disease, and liver disease.

In healthy individuals, albumin is filtered in the kidneys. When albumin is not properly filtered in the kidneys, albumin is excreted in urine. It is known that, for example, in a patient with diabetic nephropathy that is one of complications of diabetes, the amount of albumin contained in urine increases. Therefore, the urinary albumin value is an index for diagnosing diabetic nephropathy.

A urine test paper method and a turbidimetric immunoassay (a TIA method) are known as methods for detecting albumin. A detection method utilizing a reaction of albumin with a fluorescent organic compound has also been reported. For example, Nagasaki University Graduate School of Biomedical Sciences, “Tounyoubyousei Jinshou Souki Hakken wo Kanou ni Suru Shinki On Saito Sukuriiningu Hou no Kaihatsu-Koureisha Jinkou Touseki Dounyuu Suu no Genshou to Iryouhi Sakugen wo Mezashite-” (Development of a novel on-site screening method that enables early detection of diabetic nephropathy-aiming at reduction in the number of dialysis induction in the elderly and reduction in medical cost-), Mitsui Sumitomo Insurance Welfare Foundation, Report of Research Results, Vol. 17, Research Grant 2011 (issued in July 2013), available online (URL: https://www.ms-ins.com/welfare/document/list/list2011.htm, https://www.ms-ins.com/welfare/document/list/pdf/2011/4_1_04.pdf) discloses that 4-[4-(4-dimethylaminophenyl)-5-phenyl-1H-imidazol-2-yl]benzoic acid methyl ester (DAPIM) has a fluorescence property that the fluorescence of DAPIM is increased by specific binding to human serum albumin (HSA), and can be used for the detection of diabetic nephropathy.

### CITATION LIST

Non-patent Literature 1: Nagasaki University Graduate School of Biomedical Sciences, “Tounyoubyousei Jinshou Souki Hakken wo Kanou ni Suru Shinki On Saito Sukuriiningu Hou no Kaihatsu-Koureisha Jinkou Touseki Dounyuu Suu no Genshou to Iryouhi Sakugen wo Mezashite—” (Development of a novel on-site screening method that enables early detection of diabetic nephropathy-aiming at reduction in the number of dialysis induction in the elderly and reduction in medical cost −), Mitsui Sumitomo Insurance Welfare Foundation, Report of Research Results, Vol. 17, Research Grant 2011 (issued in July 2013), available online (URL: https://www.ms-ins.com/welfare/document/list/list2011.htm, https://www.ms-ins.com/welfare/document/list/pdf/2011/4_1_04.pdf)

### SUMMARY

However, the method for detecting albumin in Nagasaki University Graduate School of Biomedical Sciences, “Development of a novel on-site screening method that enables early detection of diabetic nephropathy-aiming at reduction in the number of dialysis induction in the elderly and reduction in medical cost −”, Mitsui Sumitomo Insurance Welfare Foundation, Report of Research Results, Vol. 17, Research Grant 2011 (issued in July 2013), available online (URL: https://www.ms-ins.com/welfare/document/list/list2011.htm) has room for improvement from the viewpoint of sensitivity. Therefore, an object of one aspect of the present disclosure is to provide a reagent for detecting albumin, a method for detecting albumin in a sample, a device for detecting albumin, or the like.

One aspect of the present disclosure relates to a reagent for detecting albumin, the reagent containing a terbium complex represented by the following Formula (1):

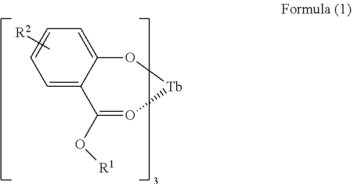

wherein R¹ represents an alkyl group having 1 to 3 carbon atoms, and R² represents a hydrogen atom, a methyl group, a methoxy group, a fluorine atom, or a hydroxy group.

In addition, one aspect of the present disclosure relates to a method for detecting albumin in a sample using the terbium complex represented by Formula (1).

One aspect of the present disclosure can provide a reagent for detecting albumin. In addition, one aspect of the present disclosure can provide a method for detecting albumin in a sample, a device for detecting albumin, or the like.

### BRIEF DESCRIPTION OF THE DRAWINGS

FIG. 1A is a diagram showing an example of an FTIR spectrum of the terbium complex according to the present disclosure;

FIG. 1B is a diagram showing an example of an FTIR spectrum of the terbium complex according to the present disclosure;

FIG. 2 is a diagram showing an example of comparison between a fluorescence spectrum curve of a reactant of the terbium complex according to the present disclosure and albumin and a fluorescence spectrum curve of the terbium complex alone;

FIG. 3 is an example of a photograph obtained by observing fluorescence emission of a reactant of the terbium complex according to the present disclosure and albumin;

FIG. 4 is a diagram showing an example of comparison between a fluorescence spectrum curve of a reactant of the terbium complex according to the present disclosure and albumin and a fluorescence spectrum curve of the terbium complex alone;

FIG. 5 is a diagram showing an example of comparison between a fluorescence spectrum curve of a reactant of the terbium complex according to the present disclosure and albumin and a fluorescence spectrum curve of the terbium complex alone;

FIG. 6 is a diagram showing an example of comparison between a fluorescence spectrum curve of a reactant of the terbium complex according to the present disclosure and albumin and a fluorescence spectrum curve of the terbium complex alone;

FIG. 7 is an example of a photograph obtained by observing fluorescence emission of a reactant of the terbium complex according to the present disclosure and albumin;

FIG. 8 is a diagram showing an example of comparison between a fluorescence spectrum curve of a reactant of the terbium complex according to the present disclosure and albumin and a fluorescence spectrum curve of the terbium complex alone;

FIG. 9 is a diagram showing an example of comparison between a fluorescence spectrum curve of a reactant of the terbium complex according to the present disclosure and albumin and a fluorescence spectrum curve of the terbium complex alone;

FIG. 10 is a diagram showing an example of comparison between a fluorescence spectrum curve of a reactant of the terbium complex according to the present disclosure and albumin and a fluorescence spectrum curve of the terbium complex alone;

FIG. 11 is a diagram showing an example of comparison between a fluorescence spectrum curve of a reactant of the terbium complex according to the present disclosure and albumin and a fluorescence spectrum curve of the terbium complex alone;

FIG. 12 is a diagram showing an example of comparison between a fluorescence spectrum curve of a reactant of the terbium complex according to the present disclosure and albumin and a fluorescence spectrum curve of the terbium complex alone;

FIG. 13 is a diagram showing an example of comparison between a fluorescence spectrum curve of a reactant of the terbium complex according to the present disclosure and albumin and a fluorescence spectrum curve of the terbium complex alone;

FIG. 14 is an example of a photograph obtained by observing fluorescence emission of a reactant of the terbium complex according to the present disclosure and albumin;

FIG. 15 is a diagram showing an example of comparison between a fluorescence spectrum curve of a reactant of the terbium complex according to the present disclosure and albumin and a fluorescence spectrum curve of the terbium complex alone;

FIG. 16 is an example of a photograph obtained by observing fluorescence emission of a reactant of the terbium complex according to the present disclosure and albumin;

FIG. 17 is a diagram showing an example of comparison between a fluorescence spectrum curve of a reactant of the terbium complex according to the present disclosure and albumin and a fluorescence spectrum curve of the terbium complex alone; and

FIG. 18 is an example of a photograph obtained by observing fluorescence emission of a reactant of the terbium complex according to the present disclosure and albumin.

### EXAMPLE EMBODIMENTS

Hereinafter, embodiments for carrying out the present disclosure will be described, but the example embodiments according to the present disclosure are not limited to the following embodiments.

### <Reagent for Detecting Albumin>

A reagent for detecting albumin of the present disclosure (also simply referred to as “reagent”) contains a terbium complex represented by Formula (1) (also simply referred to as “terbium complex” or “terbium complex of Formula (1)”).

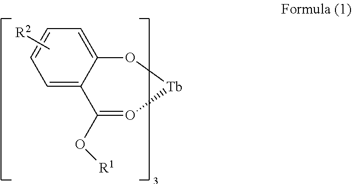

In Formula (1), R¹ represents an alkyl group having 1 to 3 carbon atoms, and R² represents a hydrogen atom, a methyl group, a methoxy group, a fluorine atom, or a hydroxy group.

In Formula (1), R¹ is an alkyl group having 1 to 3 carbon atoms, and is preferably a methyl group, an ethyl group, a propyl group, or an isopropyl group.

In Formula (1), R² represents a hydrogen atom, a methyl group, a methoxy group, a fluorine atom, or a hydroxy group. When R² is other than a hydrogen atom, the position of R² bonded to a benzene ring is not limited, but R² is preferably bonded to, for example, the 4-position of the benzene ring.

Examples of the terbium complex of Formula (1) include terbium complexes Tb-1 to Tb-7 synthesized in Synthesis Examples 1 to 7 described later. In one aspect, the terbium complex Tb-1 (a terbium complex synthesized using methyl salicylate as a raw material) and the terbium complex Tb-5 (a terbium complex synthesized using ethyl salicylate as a raw material) are particularly suitable for detecting albumin, but the terbium complex is not limited thereto.

In the present disclosure, the term “reagent” is defined as a chemical substance used for detection or quantification of a substance by chemical methods, experiments of synthesis of a substance, or measurement of physical properties. The reagent of the present disclosure may contain one terbium complex alone, or may contain two or more terbium complexes in combination.

The reagent of the present disclosure may contain a solvent in addition to the terbium complex of Formula (1). The solvent is not limited, but is preferably an organic solvent capable of dissolving the terbium complex, and examples of the solvent include dimethyl sulfoxide, methanol, ethanol, N,N-dimethylformamide, tetrahydrofuran, acetone, acetonitrile, and 1,4-dioxane. The solvent may be of one kind or a mixture of two or more kinds.

In one aspect, the reagent of the present disclosure may further contain at least one selected from a glycol-based compound and glycerol (also referred to as “glycol-based compound or the like”) as an additive in addition to the terbium complex of Formula (1). The addition of a glycol-based compound or the like promotes the reaction of the terbium complex in the reagent with albumin in the sample. Examples of the glycol-based compound include ethylene glycol, diethylene glycol, triethylene glycol, and polyethylene glycol. The content of the glycol-based compound or the like in the reagent may be 0 vol %, but may be preferably equal to or more than 3 vol %, equal to or more than 5 vol %, equal to or more than 10 vol %, equal to or more than 15 vol %, or equal to or more than 20 vol %, and may be equal to or less than 50 vol %, equal to or less than 40 vol %, equal to or less than 30 vol %, or equal to or less than 25 vol % relative to the total amount of the solvent.

In one aspect, when the terbium complex is incorporated into a solid medium such as filter paper (for example, when a test paper is produced), a reagent containing a glycol-based compound or the like is preferably used. When a test paper is produced using a reagent containing a glycol-based compound or the like, the test paper is impregnated with the glycol-based compound or the like. When albumin is detected using a test paper or the like impregnated with a receptor (the terbium complex), the impregnation of the test paper such as filter paper with a water-soluble glycol-based compound and/or glycerol as described above promotes the impregnation of the filter paper with a sample of an albumin-containing aqueous solution, and promotes the reaction of the receptor with albumin.

When a solution in which the terbium complex of Formula (1) is dissolved in a solvent is used as a reagent, the concentration of the terbium complex represented by Formula (1) in the solution (the concentration before mixing with the sample) is not limited, but may be, for example, preferably equal to or more than 0.1 mM, equal to or more than 0.2 mM, equal to or more than 0.5 mM, equal to or more than 1.0 mM, equal to or more than 1.5 mM, or equal to or more than 2.0 mM, and the upper limit of the concentration may be preferably equal to or less than 10.0 mM, equal to or less than 8.0 mM, equal to or less than 6.0 mM, or equal to or less than 5.0 mM. In the present specification, the concentration unit “M” means “mol/L”. In one aspect, when a test paper that contains the terbium complex of Formula (1) is used as a reagent, the test paper (for example, filter paper) may be impregnated with a solution of the terbium complex and then dried under a temperature condition from room temperature to 80° C. to obtain the test paper containing the terbium complex.

One aspect of the present disclosure relates to the terbium complex represented by Formula (1), and preferably relates to the terbium complex represented by Formula (1) for detecting albumin. One aspect of the present disclosure also relates to a kit for detecting albumin containing the terbium complex represented by Formula (1). One aspect of the present disclosure relates to a kit for detecting albumin containing the reagent.

The reagent for detecting albumin of the present disclosure contains the terbium complex of Formula (1), and in fluorescence intensity measurement at a specific wavelength, albumin in the sample can be detected because the intensity of fluorescence emitted from a mixture of the terbium complex and albumin (also referred to as a “reactant”) is larger than the fluorescence intensity of the terbium complex alone.

In the present disclosure, albumin may be, but not limited to, human serum albumin (HSA), bovine serum albumin (BSA), or the like.

### (Method for Producing Terbium Complex)

The terbium complex of Formula (1) can be synthesized, for example, by reacting a compound represented by Formula (21) with terbium chloride hexahydrate in water.

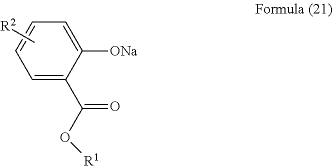

In Formula (21), R¹ represents an alkyl group having 1 to 3 carbon atoms, and R² represents a hydrogen atom, a methyl group, a methoxy group, a fluorine atom, or a hydroxy group.

The compound represented by Formula (21) is obtained, for example, by reacting a compound that has a salicylic acid ester backbone relevant to the backbone of the compound represented by Formula (21) with sodium hydroxide.

For example, when the terbium complex of Formula (1) wherein R¹ is a methyl group and R² is a hydrogen atom is synthesized, methyl salicylate and sodium hydroxide are reacted to synthesize a sodium salt of methyl salicylate (a compound according to Formula (21)), and subsequently the sodium salt of methyl salicylate and terbium chloride hexahydrate are reacted in water to obtain the target terbium complex.

### <Method for Detecting Albumin in Sample>

In one aspect of the present disclosure, a method for detecting albumin in a sample includes a step of detecting fluorescence emission of a mixture of the terbium complex represented by Formula (1) and albumin. In one preferred aspect, the method for detecting albumin in a sample includes: (i) obtaining a mixture of a sample and a reagent containing the terbium complex represented by Formula (1) described above (step (i)), (ii) irradiating the mixture with excitation light having a specific wavelength (step (ii)), and (iii) detecting fluorescence emitted from the mixture (step (iii)). Step (i) is preferably carried out in water. The method for detecting albumin of the present disclosure utilizes a phenomenon in which the fluorescence emission increases due to the interaction between the terbium complex represented by Formula (1) and albumin that are mixed and brought into contact with each other. While the terbium complex alone exhibits little fluorescence emission in an aqueous solution, the mixture (reactant) obtained by the reaction of the terbium complex with albumin newly exhibits fluorescence emission. Albumin in the sample can be detected by utilizing this phenomenon. In addition, a sample mixed with the terbium complex represented by Formula (1) exhibits little or no fluorescence emission in the absence of albumin even when the sample contains ions (sodium ion, potassium ion, calcium ion, and the like), nitrogen compounds (urea, sodium urate, creatinine, and the like), and the like that are usually present in urine. On the other hand, the sample reacts with the terbium complex and exhibits remarkable fluorescence emission in the presence of albumin when the sample contains the ions and the nitrogen compounds. Therefore, the terbium complex represented by Formula (1) is effective for detecting albumin in urine.

A urine test paper method, a turbidimetric immunoassay (a TIA method), and the like have been known as a method for detecting albumin. Although the known urine test paper method is inexpensive and can easily detect albumin, the urine test paper method may not be sensitive enough, and may not detect the histological change of the kidney due to, for example, diabetic nephropathy, even when the histological change of the kidney has already progressed. In addition, the method for detecting albumin by the TIA method tends to have high sensitivity, but is expensive and has complicated detection work. Therefore, there has been a demand for development of a reagent for detecting albumin in a sample, which can be used simply, is inexpensive, and can detect albumin with high sensitivity even when the concentration of albumin is low.

The method for detecting albumin of the present disclosure is inexpensive, can be carried out easily, and can detect albumin in a sample with high sensitivity even when the concentration of albumin in the sample is low. Therefore, the method for detecting albumin of the present disclosure is considered to be useful also for early detection, prevention, deceleration of the progression, treatment, and the like of diabetic nephropathy or the like in which the amount of albumin in urine increases.

In one aspect of the present disclosure, the sample is not limited, but is preferably a biological sample, and examples of the sample include blood, plasma, serum, lymph, saliva, sweat, tears, and urine, and in one aspect, the sample is preferably urine. The biological sample is preferably derived from a mammal, but is not limited thereto, and examples of the mammal include human, cow, goat, sheep, pig, monkey, dog, cat, rat, mouse, hamster, and guinea pig, and the mammal is more preferably human. The sample may be a solid or a solution. The solution may be body fluid, may contain a solvent that is not body fluid, or may be a mixture thereof. The solution may be a collected sample used as it is, or may be a liquid obtained by diluting the collected sample with water or the like, or concentrating the collected sample. In one aspect, the solution may be a solution used for sample measurement, a solution used for measurement for calibration, or a standard solution or a calibration solution.

The mixing (contacting) of a sample and a reagent in step (i) is preferably performed in the presence of albumin under the condition that albumin and the terbium complex can react, more preferably performed in a solution, and still more preferably performed in an aqueous solution. In one aspect, preferably, a solution containing the terbium complex of Formula (1) and the sample (preferably containing water) are mixed, stirred as necessary, and reacted at room temperature (about 5 to 40° C., preferably about 15 to 35° C.) for equal to or more than 30 seconds, and preferably equal to or more than 1 minute (for example, 1 to 120 minutes, and preferably 1 to 60 minutes).

The mixing ratio of the reagent containing the terbium complex to the sample is not limited, and may be appropriately adjusted according to the type of the terbium complex to be used, the concentration of albumin in the sample, and the like. In one aspect, for example, the reagent and the sample are preferably mixed in such a way that the amount of the terbium complex is preferably equal to or more than 1×10⁻⁶ mol, and more preferably equal to or more than 1×10⁻⁵ mol relative to 1 mg of albumin. In one aspect, when the sample and the reagent are mixed in step (i), the volume of the sample (an aqueous solution) may be, for example, equal to or more than 30 times, equal to or more than 50 times, or equal to or more than 80 times the volume of the solution (the reagent) containing the terbium complex, and the reagent and the sample may be mixed in such a way that the volume of the sample is equal to or less than 500 times, equal to or less than 300 times, or equal to or less than 200 times the volume of the solution (the reagent) containing the terbium complex, but the mixing ratio is not limited thereto. In one aspect, for example, the the reagent and the sample may be mixed in such a way that {the volume of the reagent containing the terbium complex (preferably a solution having a concentration of 0.1 mM to 10 mM)}:{the volume of the sample (an aqueous solution)} is 1:30 to 1:200, preferably 1:80 to 1:150.

In one aspect, the reaction of a reagent containing the terbium complex of Formula (1) with a sample may be performed in a solid medium containing the reagent. The solid medium may be, for example, paper (for example, filter paper), glass (for example, glass fiber and porous glass substrate), a resin (for example, polymethyl methacrylate, polyethylene, polypropylene, polyvinyl chloride, polystyrene, nylon resin, polyamide, polycarbonate, polyethylene terephthalate, polybutylene terephthalate, and polyphenylene oxide), or a water-soluble polymer (cellulose-based polymer, agarose, starch-based polymer, sodium alginate, acrylic acid-based polymer, acrylamide-based polymer, polyvinyl alcohol, polyethylene oxide, polyvinyl pyrrolidone, and the like) but the solid medium is not limited thereto. One aspect of the method for detecting albumin in a solid medium is, for example, a method using a test paper containing the terbium complex. The test paper containing the terbium complex can be obtained, for example, by impregnating paper such as filter paper with a solution of the terbium complex and then drying the paper. The condition for impregnating paper such as filter paper with the solution of the terbium complex and then drying the paper is not limited, but for example, the temperature is preferably equal to or more than room temperature (15° C. to 30° C.), more preferably equal to or more than 40° C., still more preferably equal to or more than 50° C., and preferably equal to or less than 80° C. The relative humidity during drying is preferably 15% RH to 60% RH, and the environmental pressure is preferably equal to or less than standard atmospheric pressure (1013.25 hPa), but is not limited thereto. The drying time is not limited, but may be, for example, equal to or more than one hour. In one aspect, the solution of the terbium complex to be impregnated into paper such as filter paper preferably contains at least one selected from the group consisting of the glycol-based compound and glycerol described above. The incorporation of the glycol-based compound and/or glycerol that are water-soluble compounds in paper such as filter paper promotes the impregnation of the filter paper with an aqueous solution (an albumin aqueous solution) of the sample, and promotes the reaction of the terbium complex with albumin.

One aspect of the method for detecting albumin in a solid medium includes immersing a test paper containing the terbium complex in an aqueous solution of a sample containing albumin to cause a reaction, and detecting fluorescence emitted from a reactant of the terbium complex and albumin. Another aspect of the method for detecting albumin in a solid medium includes adding dropwise an aqueous solution of a sample containing albumin to a test paper containing the terbium complex to cause a reaction, and detecting fluorescence emitted from a reactant of the terbium complex and albumin.

In step (ii), the mixture obtained in step (i) is irradiated with excitation light. The wavelength of the excitation light to be applied is not limited, and is, for example, preferably 200 to 500 nm, and more preferably 300 to 400 nm.

In step (iii), while the terbium complex alone exhibits little fluorescence emission, a reactant of the terbium complex and albumin newly exhibits fluorescence emission, and albumin can be detected by utilizing this phenomenon. In one aspect, determining the concentration of albumin by comparing the intensity of the detected fluorescence to a predetermined reference value may also be performed. In one aspect, the concentration of albumin in a sample may be determined from an image data showing the fluorescence emission of a reaction product between the sample and the terbium complex. Specifically, first, each of a plurality of solutions of albumin having known and different concentrations is reacted with the terbium complex, and the reaction product is irradiated with excitation light, and image data showing their light-emitting states are obtained in advance as reference image data. Next, a sample of unknown concentration is reacted with the terbium complex, and its light-emitting state is photographed in the same manner as the reference image data, to obtain a sample image data. Then, by comparing the obtained sample image data with the reference image data, the albumin concentration can be determined. By incorporating such a system for collating image data into a smartphone, a single smartphone can obtain light-emitting image data of a sample, subsequently compare the image data of the sample with the reference image data, and determine the albumin concentration in a sample. From the viewpoint of accurately photographing the intensity of the light-emitting images, it is preferable to photograph them in a state where no external light enters, for example, in the absence of external illumination.

The terbium complex of Formula (1) of the present disclosure can detect albumin in a sample with high sensitivity even when the concentration of albumin in the sample is low. The concentration of albumin in the sample may be, for example, equal to or more than 0.0001 mg/mL, equal to or more than 0.0005 mg/mL, equal to or more than 0.001 mg/mL, or equal to or more than 0.01 mg/mL, and may be, for example, equal to or less than 100 mg/mL, equal to or less than 50 mg/mL, equal to or less than 10 mg/mL, equal to or less than 5 mg/mL, or equal to or less than 3 mg/mL. The terbium complex of the present disclosure can detect the presence of albumin with high sensitivity even when the concentration of albumin in the sample is low (for example, the concentration of albumin may be less than 0.3 mg/mL, equal to or less than 0.1 mg/mL, equal to or less than 0.01 mg/mL, or less than 0.01 mg/mL).

### <Device for Detecting Albumin>

One aspect of the present disclosure relates to a device for detecting albumin (also simply referred to as “detection device” or “albumin detection device”) containing a reagent containing the terbium complex represented by Formula (1). In one aspect, the albumin detection device includes a capturing unit for albumin having a reagent containing the terbium complex represented by Formula (1), and a detection unit for detecting that albumin has been captured in the capturing unit.

### (Capturing Unit)

The capturing unit of the albumin detection device of the present disclosure contains a reagent containing the terbium complex of Formula (1). For the reagent, the description of the reagent for detecting albumin described above applies. In one aspect, the reagent contains a solvent (preferably an organic solvent), and is preferably a solution in which the terbium complex of Formula (1) is dissolved. In one aspect, the sample and the reagent may be mixed in the capturing unit.

### (Detection Unit)

The detection unit of the albumin detection device is preferably configured to be capable of optically detecting that albumin has been captured in the capturing unit. The detection unit may be configured as a separate device from the capturing unit rather than as a device integrated with the capturing unit. In one aspect, in order to detect the fluorescence emission of a reactant of the terbium complex of Formula (1) and albumin, the optical detection unit includes an excitation light source (a light emitting unit) and a detection element (a light receiving unit for fluorescence), and can detect and/or measure the concentration of albumin based on the observed change in fluorescence intensity.

In one aspect, the detection unit may include a computer that executes a program for processing the detection and/or the concentration measurement of albumin. Such a program may be, for example, a program that causes a computer to execute the stages of (i) receiving a signal from an optical detection element, (ii) analyzing the received signal to determine the presence or absence and/or the concentration of albumin, and (iii) outputting an analysis result.

In one aspect of the present disclosure, the analysis of the received signal may include determining the presence or absence and/or the concentration of albumin, for example, by comparing the received signal to a predetermined reference value. Furthermore, in one aspect of the present disclosure, the analysis result can be output to, for example, a display device connected to a sensor (a detection device), or another device connected via a network.

### EXAMPLES

Hereinafter, the present disclosure will be described more specifically with reference to examples, but the present disclosure is not limited to these examples.

### Synthesis Example 1: Synthesis of Terbium Complex (Tb-1) Represented by the Following Formula

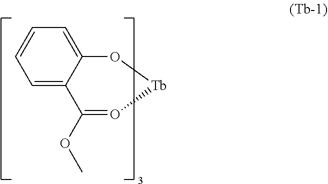

In 5 ml of acetone, 2.254 g of methyl salicylate was dissolved, then a solution obtained by dissolving 0.595 g of sodium hydroxide in 10 ml of water was added thereto, and the mixture was stirred at room temperature overnight. The solvent was distilled off under reduced pressure and dried under reduced pressure to obtain 2.265 g of a Na salt of methyl salicylate. Then, 0.4632 g of Na salt of methyl salicylate was dissolved in 20 ml of water, then a solution obtained by dissolving 0.3 g of terbium chloride hexahydrate in 20 ml of water was added thereto, and the mixture was stirred at room temperature overnight. A precipitated terbium complex was filtered off, washed with water, and then dried under reduced pressure to obtain 0.399 g of the target white terbium complex Tb-1.

The results of the elemental analysis of the obtained terbium complex were as follows: carbon: 46.6% (theoretical value: 47.1%) and hydrogen: 3.2% (theoretical value: 3.5%).

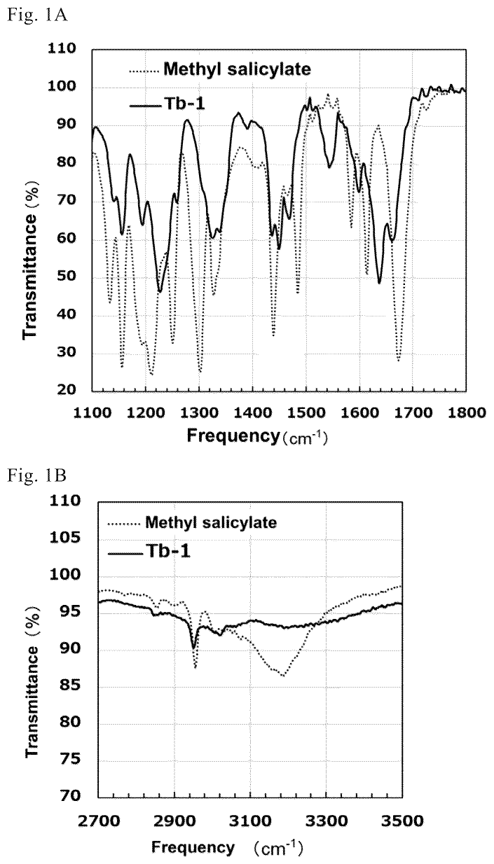

In addition, the FTIR spectrum of Tb-1 was measured. The obtained FTIR spectrum is shown in FIG. 1A and FIG. 1B. The characteristic absorption of an OH group in methyl salicylate in the raw material (3000 to 3300-1) disappeared (FIG. 1B), and furthermore, the characteristic absorption of C═O in methyl salicylate was 1673 cm⁻¹, whereas the characteristic absorption of C═O in Tb-1 shifted to the low frequency side and was 1660 cm⁻¹ (FIG. 1A). Furthermore, the characteristic absorption of Ph-O in methyl salicylate was 1251 cm⁻¹, whereas the characteristic absorption of Ph-O in Tb-1 also shifted to the low frequency side and was 1227 cm⁻¹ (FIG. 1A). This shows that Tb-1 has a structural formula in which a carbonyl group and a Ph-O group are coordinated to terbium.

### Synthesis Example 2: Synthesis of Terbium Complex (Tb-2) Represented by the Following Formula

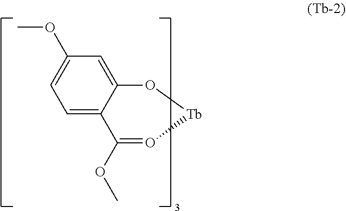

A terbium complex Tb-2 was synthesized in the same manner as in Synthesis Example 1 except that methyl 4-methoxysalicylate was used instead of methyl salicylate. The FTIR spectrum of Tb-2 was measured. The characteristic absorption of C═O in methyl 4-methoxysalicylate was 1660 cm⁻¹, whereas the characteristic absorption of C═O in Tb-2 shifted to the low frequency side and was 1632 cm⁻¹. Furthermore, the characteristic absorption of Ph-O in methyl 4-methoxysalicylate was 1247 cm⁻¹, whereas the characteristic absorption of Ph-O in Tb-2 also shifted to the low frequency side and was 1234 cm⁻¹. This shows that Tb-2 has a structural formula in which a carbonyl group and a Ph-O group are coordinated to terbium.

### Synthesis Example 3: Synthesis of Terbium Complex (Tb-3) Represented by the Following Formula

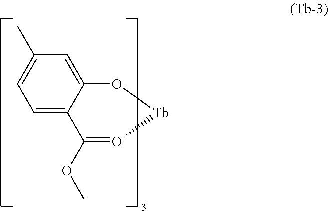

A terbium complex Tb-3 was synthesized in the same manner as in Synthesis Example 1 except that methyl 4-methylsalicylate was used instead of methyl salicylate. The FTIR spectrum of Tb-3 was measured. The characteristic absorption of C═O in methyl 4-methylsalicylate was 1660 cm⁻¹, whereas the characteristic absorption of C═O in Tb-3 shifted to the low frequency side and was 1633 cm⁻¹. In addition, the characteristic absorption of Ph-O in methyl 4-methylsalicylate was 1247 cm⁻¹, whereas the characteristic absorption of Ph-O in Tb-3 also shifted to the low frequency side and was 1234 cm⁻¹. This shows that Tb-3 has a structural formula in which a carbonyl group and a Ph-O group are coordinated to terbium.

### Synthesis Example 4: Synthesis of Terbium Complex (Tb-4) Represented by the Following Formula

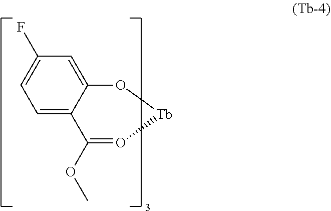

A terbium complex Tb-4 was synthesized in the same manner as in Synthesis Example 1 except that methyl 4-fluorosalicylate was used instead of methyl salicylate. The FTIR spectrum of Tb-4 was measured. The characteristic absorption of C═O in methyl 4-fluorosalicylate was 1665 cm⁻¹, whereas the characteristic absorption of C═O in Tb-4 shifted to the low frequency side and was 1640 cm⁻¹. In addition, the characteristic absorption of Ph-O in methyl 4-fluorosalicylate was 1258 cm⁻¹, whereas the characteristic absorption of Ph-O in Tb-4 also shifted to the low frequency side and was 1227 cm⁻¹. This shows that Tb-4 has a structural formula in which a carbonyl group and a Ph-O group are coordinated to terbium.

### Synthesis Example 5: Synthesis of Terbium Complex (Tb-5) Represented by the Following Formula

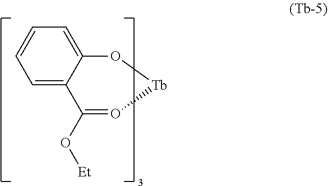

A terbium complex Tb-5 was synthesized in the same manner as in Synthesis Example 1 except that ethyl salicylate was used instead of methyl salicylate (in the formula, Et represents an ethyl group). The FTIR spectrum of Tb-5 was measured. The characteristic absorption of C═O in ethyl salicylate was 1671 cm⁻¹, whereas the characteristic absorption of C═O in Tb-5 shifted to the low frequency side and was 1658 cm⁻¹. In addition, the characteristic absorption of Ph-O in ethyl salicylate was 1248 cm⁻¹, whereas the characteristic absorption of Ph-O in Tb-5 also shifted to the low frequency side and was 1225 cm⁻¹. This shows that Tb-5 has a structural formula in which a carbonyl group and a Ph-O group are coordinated to terbium.

### Synthesis Example 6: Synthesis of Terbium Complex (Tb-6) Represented by the Following Formula

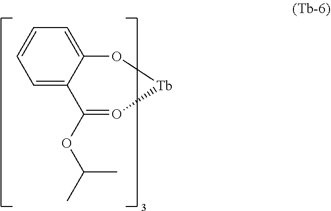

A terbium complex Tb-6 was synthesized in the same manner as in Synthesis Example 1 except that isopropyl salicylate was used instead of methyl salicylate. The FTIR spectrum of Tb-6 was measured. The characteristic absorption of C═O in isopropyl salicylate was 1664 cm⁻¹, whereas the characteristic absorption of C═O in Tb-6 shifted to the low frequency side and was 1656 cm⁻¹. In addition, the characteristic absorption of Ph-O in isopropyl salicylate was 1250 cm⁻¹, whereas the characteristic absorption of Ph-O in Tb-6 also shifted to the low frequency side and was 1227 cm⁻¹. This shows that Tb-6 has a structural formula in which a carbonyl group and a Ph-O group are coordinated to terbium.

### Synthesis Example 7: Synthesis of Terbium Complex (Tb-7) Represented by the Following Formula

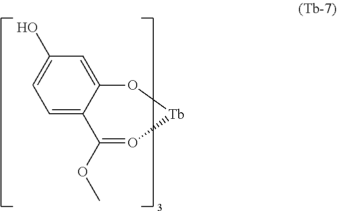

A terbium complex Tb-7 was synthesized in the same manner as in Synthesis Example 1 except that methyl 4-hydroxysalicylate was used instead of methyl salicylate. The FTIR spectrum of Tb-7 was measured. The characteristic absorption of C═O in methyl 4-hydroxysalicylate was 1636 cm⁻¹, whereas the characteristic absorption of C═O in Tb-7 shifted to the low frequency side and was 1628 cm⁻¹. In addition, the characteristic absorption of Ph-O in methyl 4-hydroxysalicylate was 1264 cm⁻¹, whereas the characteristic absorption of Ph-O in Tb-7 also shifted to the low frequency side and was 1254 cm⁻¹. This shows that Tb-7 has a structural formula in which a carbonyl group and a Ph-O group are coordinated to terbium.

### Example 1

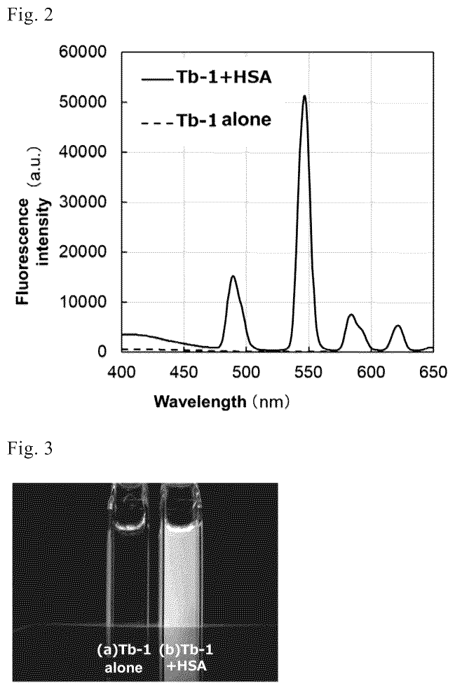

A dimethyl sulfoxide (DMSO) solution of the terbium complex (Tb-1) obtained in Synthesis Example 1 was prepared (concentration: 5 mM), and an aqueous solution of albumin (HSA, derived from human serum, manufactured by FUJIFILM Wako Pure Chemical Corporation) was further prepared (concentration: 1 mg/ml). Then, 40 μL of a DMSO solution of Tb-1 was added to 4 ml of an aqueous HSA solution, and the mixture was allowed to stand at room temperature for 1 hour. Thereafter, the mixture was filtered off using a 0.45 micron PTFE filter, 3 ml of the filtrate was placed in a quartz cell, and the fluorescence spectrum with excitation light of 350 nm was measured. In addition, 40 μL of a DMSO solution of Tb-1 was added to 4 ml of water containing no HSA, and the fluorescence spectrum was measured in the same manner. The obtained fluorescence spectrum curves are shown in FIG. 2. The solid line represents the fluorescence spectrum of Tb-1+HSA, and the broken line represents the fluorescence spectrum of Tb-1 alone. The fluorescence intensity of Tb-1+HSA at a wavelength of 546 nm in the obtained fluorescence spectrum was 51,200. On the other hand, it was found that the fluorescence intensity of Tb-1 alone was 50, and the fluorescence emission intensity increases to about 1,000 times. A photograph of the quartz cells containing each solution in a light-emitting state excited by a UV lamp having a wavelength of 365 nm is shown in FIG. 3. In a solution containing HSA (b), yellow-green emission was visually observed. On the other hand, in a solution containing no HSA (a), emission was not observed.

As shown in FIG. 3, it was found that the presence of HSA in a sample can be confirmed by photographing the light-emitting state of the reaction product between the terbium complex and HSA using a smartphone or the like. Furthermore, as described below, the concentration of HSA in a sample can be determined from the image data of the light-emitting state of the reaction product of the terbium complex and HSA. First, multiple aqueous solutions of HSA having known and different concentrations are respectively reacted with the terbium complex, and the reaction products are irradiated with excitation light, and image data showing their light-emitting states are obtained in advance as reference image data. Next, a sample of unknown concentration is reacted with the terbium complex, and its light-emitting state is photographed in the same manner as the reference image data to obtain a sample image data. Then, by comparing the obtained sample image data with the reference image data, the HSA concentration can be determined. By incorporating such a system for collating image data into a smartphone, a single smartphone can obtain light-emitting image data of a sample, subsequently compare the image data with the reference image data, and determine the HSA concentration in a sample. From the viewpoint of accurately photographing the intensity of the light-emitting images, it is preferable to photograph them in a state where no external light enters, for example, in the absence of external illumination.

### Examples 2 and 3

Evaluation was carried out in the same manner as in Example 1, except that each of the terbium complexes obtained in Synthesis Examples 2 and 3 (Tb-2 and Tb-3) was used instead of the terbium complex obtained in Synthesis Example 1 (Tb-1). The fluorescence intensity at 546 nm in the obtained fluorescence spectrum is shown in Table 1. From these results, it was found that the emission intensity of the terbium complexes (Tb-2 and Tb-3) increased in the presence of HSA. Note that “a. u.” represents an arbitrary unit.

TABLE 1

Fluorescence intensity at
wavelength of 546 nm (a. u.)

| | Terbium | Terbium |
|---|---|---|
| | complex + HSA | complex alone |
| Example 2 | 4067 | 22 |
| Example 3 | 6433 | 26 |

### Example 4

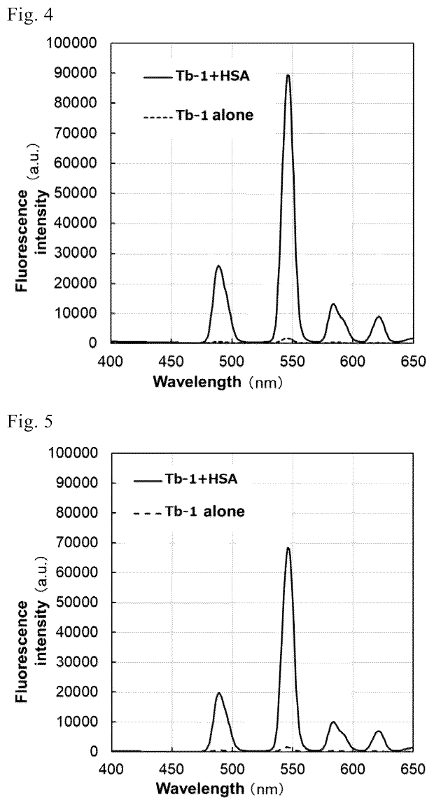

A dimethyl sulfoxide (DMSO) solution of the terbium complex (Tb-1) obtained in Synthesis Example 1 was prepared (concentration: 2.5 mM), and an aqueous solution of albumin (HSA, derived from human serum, manufactured by FUJIFILM Wako Pure Chemical Corporation) was further prepared (concentration: 0.1 mg/ml). Then, 40 μL of a DMSO solution of Tb-1 was added to 4 ml of an aqueous HSA solution, and the mixture was allowed to stand at room temperature for 1 hour. Thereafter, 3 ml of the mixed solution was placed in a quartz cell, and the fluorescence spectrum with excitation light of 350 nm was measured. In addition, 40 L of a DMSO solution of Tb-1 was added to 4 ml of water containing no HSA, and the fluorescence spectrum was measured in the same manner. The obtained fluorescence spectrum curves are shown in FIG. 4. The solid line represents the fluorescence spectrum of Tb-1+HSA, and the broken line represents the fluorescence spectrum of Tb-1 alone. It was found that the fluorescence intensity of Tb-1+HSA at a wavelength of 546 nm in the obtained fluorescence spectrum increases to about 50 times the fluorescence intensity of Tb-1 alone.

### Example 5

A dimethyl sulfoxide (DMSO) solution of the terbium complex (Tb-1) obtained in Synthesis Example 1 was prepared (concentration: 2.5 mM), and an aqueous solution of albumin (HSA, derived from human serum, manufactured by FUJIFILM Wako Pure Chemical Corporation) was further prepared (concentration: 0.01 mg/ml). Then, 40 μL of a DMSO solution of Tb-1 was added to 4 ml of an aqueous HSA solution, and the mixture was allowed to stand at room temperature for 1 hour. Thereafter, 3 ml of the mixed solution was placed in a quartz cell, and the fluorescence spectrum with excitation light of 350 nm was measured. In addition, 40 μL of a DMSO solution of Tb-1 was added to 4 ml of water containing no HSA, and the fluorescence spectrum was measured in the same manner. The obtained fluorescence spectrum curves are shown in FIG. 5. The solid line represents the fluorescence spectrum of Tb-1+HSA, and the broken line represents the fluorescence spectrum of Tb-1 alone. It was found that the fluorescence intensity of Tb-1+HSA at a wavelength of 546 nm in the obtained fluorescence spectrum increases to about 46 times the fluorescence intensity of Tb-1 alone.

### Example 6

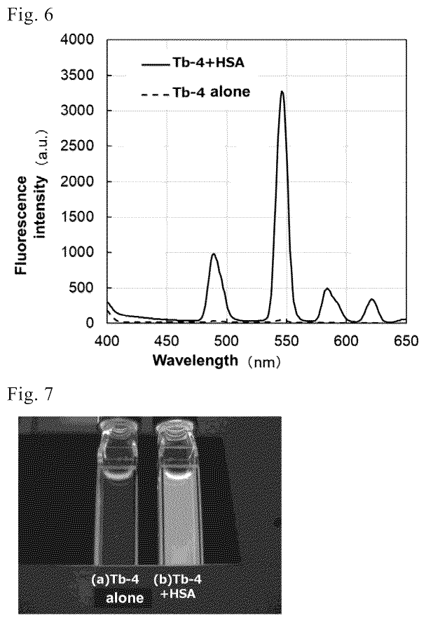

A dimethyl sulfoxide (DMSO) solution of the terbium complex (Tb-4) obtained in Synthesis Example 4 was prepared (concentration: 0.25 mM), and an aqueous solution of albumin (HSA, derived from human serum, manufactured by FUJIFILM Wako Pure Chemical Corporation) was further prepared (concentration: 0.01 mg/ml). Then, 40 μL of a DMSO solution of Tb-4 was added to 4 ml of an aqueous HSA solution, and the mixture was allowed to stand at room temperature for 1 hour. Thereafter, 3 ml of the mixed solution was placed in a quartz cell, and the fluorescence spectrum with excitation light of 350 nm was measured. In addition, 40 μL of a DMSO solution of Tb-4 was added to 4 ml of water containing no HSA, and the fluorescence spectrum was measured in the same manner. The obtained fluorescence spectrum curves are shown in FIG. 6. The solid line represents the fluorescence spectrum of Tb-4+HSA, and the broken line represents the fluorescence spectrum of Tb-4 alone. It was found that the fluorescence intensity of Tb-4+HSA at a wavelength of 546 nm in the obtained fluorescence spectrum increases to about 70 times the fluorescence intensity of Tb-4 alone. A photograph of the quartz cells containing each solution in a light-emitting state excited by a UV lamp having a wavelength of 365 nm is shown in FIG. 7. In a solution containing HSA (b), yellow-green emission was visually observed. On the other hand, in a solution containing no HSA (a), emission was not observed.

### Example 7

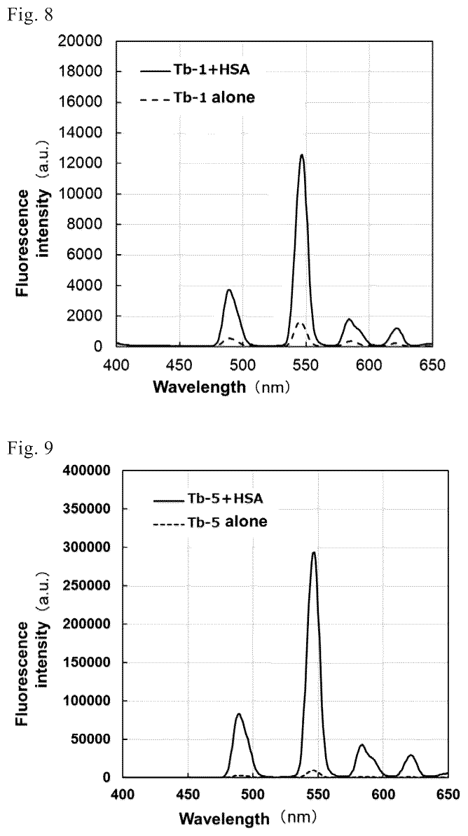

A dimethyl sulfoxide (DMSO) solution of the terbium complex (Tb-1) obtained in Synthesis Example 1 was prepared (concentration: 2.5 mM), and an aqueous solution of albumin (HSA, derived from human serum, manufactured by FUJIFILM Wako Pure Chemical Corporation) was further prepared (concentration: 0.001 mg/ml). Then, 40 μL of a DMSO solution of Tb-1 was added to 4 ml of an aqueous HSA solution, and the mixture was allowed to stand at room temperature for 1 hour. Thereafter, 3 ml of the mixed solution was placed in a quartz cell, and the fluorescence spectrum with excitation light of 350 nm was measured. In addition, 40 μL of a DMSO solution of Tb-1 was added to 4 ml of water containing no HSA, and the fluorescence spectrum was measured in the same manner. The obtained fluorescence spectrum curves are shown in FIG. 8. The solid line represents the fluorescence spectrum of Tb-1+HSA, and the broken line represents the fluorescence spectrum of Tb-1 alone. It was found that the fluorescence intensity of Tb-1+HSA at a wavelength of 546 nm in the obtained fluorescence spectrum increases to about 8 times the fluorescence intensity of Tb-1 alone.

### Example 8

A dimethyl sulfoxide (DMSO) solution of the terbium complex (Tb-5) obtained in Synthesis Example 5 was prepared (concentration: 2.5 mM), and an aqueous solution of albumin (HSA, derived from human serum, manufactured by FUJIFILM Wako Pure Chemical Corporation) was further prepared (concentration: 0.01 mg/ml). Then, 40 μL of a DMSO solution of Tb-5 was added to 4 ml of an aqueous HSA solution, and the mixture was allowed to stand at room temperature for 1 hour. Thereafter, 3 ml of the mixed solution was placed in a quartz cell, and the fluorescence spectrum with excitation light of 350 nm was measured. In addition, 40 μL of a DMSO solution of Tb-5 was added to 4 ml of water containing no HSA, and the fluorescence spectrum was measured in the same manner. The obtained fluorescence spectrum curves are shown in FIG. 9. The solid line represents the fluorescence spectrum of Tb-5+HSA, and the broken line represents the fluorescence spectrum of Tb-5 alone. It was found that the fluorescence intensity of Tb-5+HSA at a wavelength of 546 nm in the obtained fluorescence spectrum increases to about 20 times the fluorescence intensity of Tb-5 alone.

### Example 9

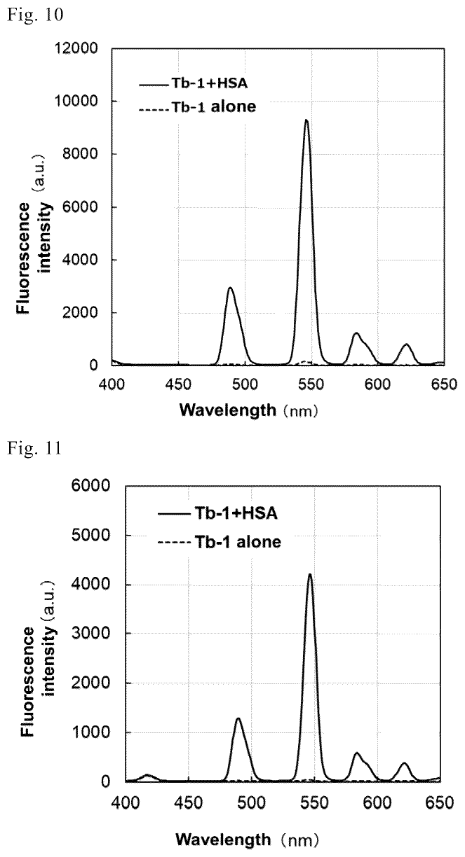

A dimethyl sulfoxide (DMSO) solution of the terbium complex (Tb-1) obtained in Synthesis Example 1 was prepared (concentration: 0.5 mM), and an aqueous solution of albumin (HSA, derived from human serum, manufactured by FUJIFILM Wako Pure Chemical Corporation) was further prepared (concentration: 0.01 mg/ml). Then, 40 μL of a DMSO solution of Tb-1 was added to 4 ml of an aqueous HSA solution, and the mixture was allowed to stand at room temperature for 1 minute. Thereafter, 3 ml of the mixed solution was placed in a quartz cell, and the fluorescence spectrum with excitation light of 350 nm was measured. In addition, 40 μL of a DMSO solution of Tb-1 was added to 4 ml of water containing no HSA, and the fluorescence spectrum was measured in the same manner. The obtained fluorescence spectrum curves are shown in FIG. 10. The solid line represents the fluorescence spectrum of Tb-1+HSA, and the broken line represents the fluorescence spectrum of Tb-1 alone. It was found that the fluorescence intensity of Tb-1+HSA at a wavelength of 546 nm in the obtained fluorescence spectrum increases to about 60 times the fluorescence intensity of Tb-1 alone.

### Example 10

Evaluation was performed in the same manner as in Example 9 except that excitation light of 365 nm was used instead of the excitation light of 350 nm. The obtained fluorescence spectrum curves are shown in FIG. 11. The solid line represents the fluorescence spectrum of Tb-1+HSA, and the broken line represents the fluorescence spectrum of Tb-1 alone. It was found that the fluorescence intensity of Tb-1+HSA at a wavelength of 546 nm in the obtained fluorescence spectrum increases to about 90 times the fluorescence intensity of Tb-1 alone.

### Example 11

### <Detection of HSA in Aqueous Solution Containing Various Ions (Sodium Ion, Potassium Ion, and Calcium Ion) and Nitrogen Compounds (Urea, Sodium Urate, and Creatinine) Present in Urine>

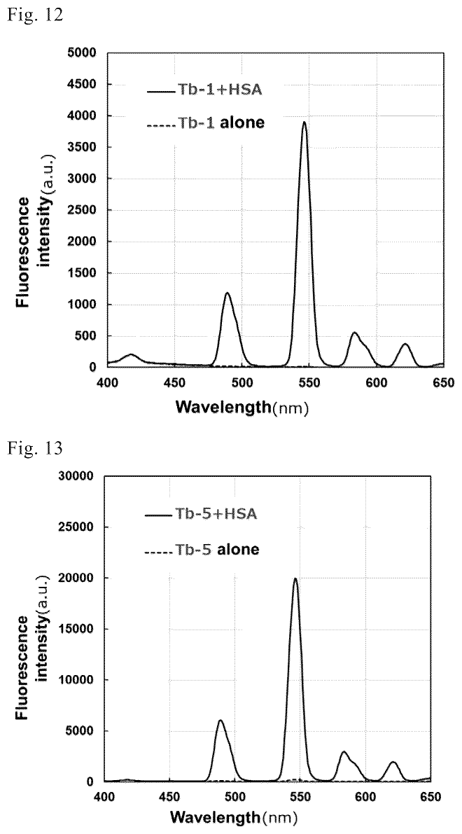

In 24.99 ml of water, 4.2 ml of a 1 M aqueous sodium chloride solution, 0.12 ml of a 1 M aqueous potassium chloride solution, 0.69 ml of a 0.1 M aqueous calcium chloride solution, 6 mg of urea, 0.3 mg of creatinine, and 1.7 mg of sodium urate were dissolved to prepare an aqueous solution (11a) containing various ions and nitrogen compounds. Then, an aqueous solution of albumin (HSA, derived from human serum, manufactured by FUJIFILM Wako Pure Chemical Corporation) (aqueous HSA solution (11b)) was prepared (concentration: 0.01 mg/ml) using the aqueous solution (11a). Then, a dimethyl sulfoxide (DMSO) solution of the terbium complex (Tb-1) obtained in Synthesis Example 1 was prepared (concentration: 0.5 mM). Then, an aqueous HSA solution (11b) was placed in a 3 ml quartz cell, 30 μL of a DMSO solution of Tb-1 was added to the quartz cell, and the quartz cell was allowed to stand at room temperature for 2 minutes. Thereafter, the fluorescence spectrum with excitation light of 365 nm was measured. In addition, 30 μL of a DMSO solution of Tb-1 was added to 3 ml of the aqueous solution containing no HSA (aqueous solution (11a)), and the fluorescence spectrum was measured in the same manner. The obtained fluorescence spectrum curves are shown in FIG. 12. The solid line represents the fluorescence spectrum of Tb-1+HSA, and the broken line represents the fluorescence spectrum of Tb-1 alone. The fluorescence intensity of Tb-1+HSA at a wavelength of 546 nm in the obtained fluorescence spectrum was 3,906. On the other hand, the fluorescence intensity of Tb-1 alone was 14. Therefore, it was found that even in a solution containing various ions and nitrogen compounds usually present in urine, the fluorescence emission intensity increases to about 279 times in the presence of HSA, and HSA can be detected by fluorescence.

### Example 12

### <Detection of HSA in Aqueous Solution Containing Various Ions (Sodium Ion, Potassium Ion, and Calcium Ion) and Nitrogen Compounds (Urea, Sodium Urate, and Creatinine) Present in Urine>

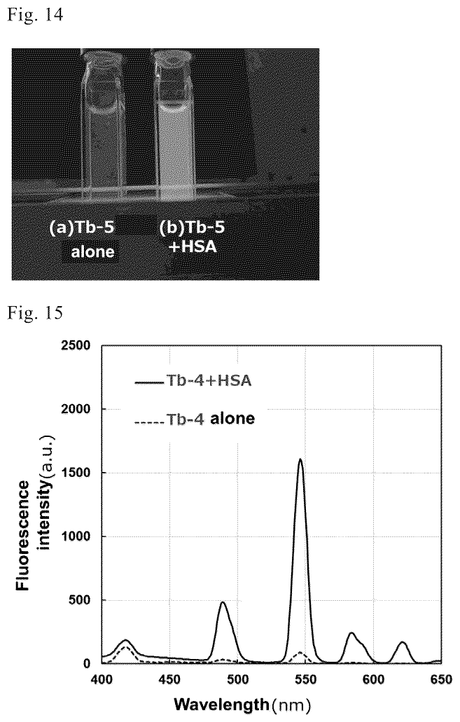

Evaluation was performed in the same manner as in Example 11 except that Tb-5 was used instead of Tb-1. The obtained fluorescence spectrum curves are shown in FIG. 13. The solid line represents the fluorescence spectrum of Tb-5+HSA, and the broken line represents the fluorescence spectrum of Tb-5 alone. The fluorescence intensity of Tb-5+HSA at a wavelength of 546 nm in the obtained fluorescence spectrum was 19,981. On the other hand, the fluorescence intensity of Tb-5 alone was 227. Therefore, it was found that the fluorescence emission intensity increases to about 88 times in the presence of HSA. A photograph of the quartz cells containing each solution in a light-emitting state excited by a UV lamp having a wavelength of 365 nm is shown in FIG. 14. In a solution containing HSA (b), yellow-green emission was visually observed. On the other hand, in a solution containing no HSA (a), emission was hardly observed.

### Example 13

### <Detection in Aqueous Solution Containing Various Ions (Sodium Ion, Potassium Ion, and Calcium Ion) and Nitrogen Compounds (Urea, Sodium Urate, and Creatinine) Present in Urine>

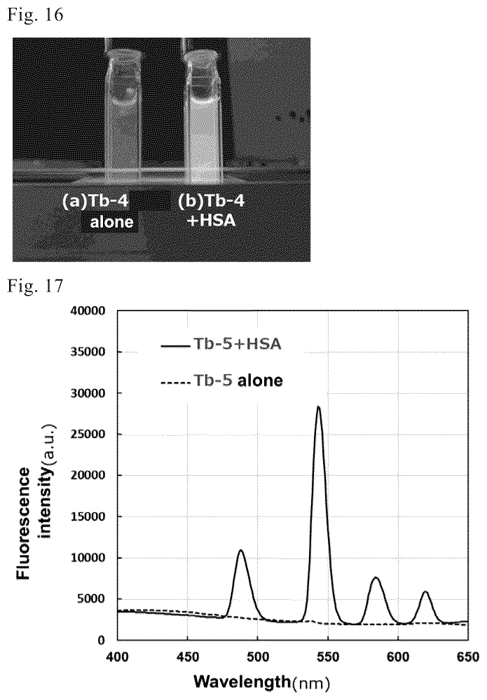

Evaluation was performed in the same manner as in Example 11 except that Tb-4 was used instead of Tb-1. The obtained fluorescence spectrum curves are shown in FIG. 15. The solid line represents the fluorescence spectrum of Tb-4+HSA, and the broken line represents the fluorescence spectrum of Tb-4 alone. The fluorescence intensity of Tb-4+HSA at a wavelength of 546 nm in the obtained fluorescence spectrum was 1,610. On the other hand, the fluorescence intensity of Tb-4 alone was 86. Therefore, it was found that the fluorescence emission intensity increases to about 19 times in the presence of HSA. A photograph of the quartz cells containing each solution in a light-emitting state excited by a UV lamp having a wavelength of 365 nm is shown in FIG. 16. In a solution containing HSA (b), yellow-green emission was visually observed. On the other hand, in a solution containing no HSA (a), emission was hardly observed.

### Example 14

<Detection of HSA with Test Paper>

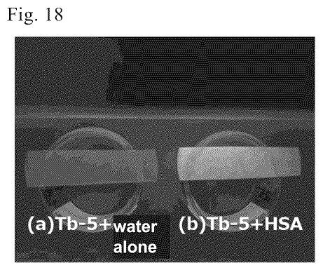

A mixed solution of the terbium complex (Tb-5) obtained in Synthesis Example 5 in dimethyl sulfoxide (DMSO) and polyethylene glycol 200 (manufactured by Tokyo Chemical Industry Co., Ltd.) (volume ratio: 80/20) was prepared (concentration: 20 mM), and 0.2 ml of the solution was added dropwise to filter paper piece (Φ: 40 mm) to impregnate the filter paper with the solution, and the filter paper was dried at 50° C. and cut into a width of 1 cm. In addition, an aqueous solution of albumin (HSA, derived from human serum, manufactured by FUJIFILM Wako Pure Chemical Corporation) was prepared (concentration: 0.1 mg/ml). Then, the filter paper impregnated with the terbium complex was immersed in 9 ml of an aqueous HSA solution in a sample container (made of glass and having a capacity of 10 ml) for 30 seconds, and the filter paper was taken out, and then allowed to stand at room temperature for 5 minutes. The fluorescence spectrum of the obtained filter paper with excitation light of 350 nm was measured. In addition, the fluorescence spectrum of the filter paper impregnated with the terbium complex and immersed in 9 ml of water containing no HSA was also measured in the same manner. The obtained fluorescence spectrum curves are shown in FIG. 17. The solid line represents the fluorescence spectrum of Tb-5+HSA, and the broken line represents the fluorescence spectrum of Tb-5+water. It was found that the fluorescence spectrum of Tb-5+water does not have a peak of emission in the vicinity of a wavelength of 540, whereas the fluorescence spectrum of Tb-5+HSA has a peak of fluorescence intensity of 28,400 at a wavelength of 543 nm. A photograph of each of the filter paper pieces impregnated with the terbium complex in a light-emitting state excited by a UV lamp having a wavelength of 365 nm is shown in FIG. 18. In the filter paper piece (b) immersed in an aqueous solution containing HSA, yellow-green emission characteristic of the terbium complex was clearly observed in the image. On the other hand, in the filter paper piece (a) immersed only in water containing no HSA, emission was hardly observed. Therefore, it was found that in the aqueous solution containing HSA, the presence of HSA can be visually observed by the emission image.

While the present disclosure has been described with reference to example embodiments and examples thereof, the present disclosure is not limited to the example embodiments and examples described above. It will be understood by those of ordinary skill in the art that various changes in form and details may be made therein without departing from the spirit and scope of the present disclosure as defined by the claims. And each embodiment can be appropriately combined with other embodiments.

Some or all of the example embodiments described above may be described as in the following supplementary notes, but the disclosure of the present application is not limited to the following supplementary notes.

### (Supplementary Note 1)

A reagent for detecting albumin, the reagent containing a terbium complex represented by Formula (1):

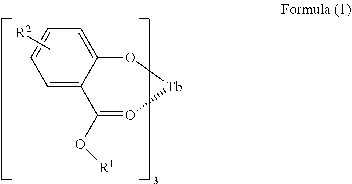

wherein R¹ represents an alkyl group having 1 to 3 carbon atoms, and R² represents a hydrogen atom, a methyl group, a methoxy group, a fluorine atom, or a hydroxy group.

### (Supplementary Note 2)

The reagent according to Supplementary Note 1, further containing a solvent.

### (Supplementary Note 3)

The reagent according to Supplementary Note 1 or 2, further containing at least one selected from the group consisting of a glycol-based compound and glycerol.

### (Supplementary Note 4)

A method for detecting albumin in a sample using a terbium complex represented by Formula (1):

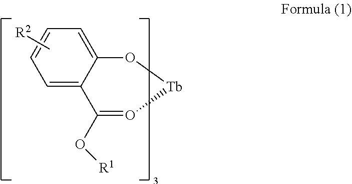

wherein R¹ represents an alkyl group having 1 to 3 carbon atoms, and R² represents a hydrogen atom, a methyl group, a methoxy group, a fluorine atom, or a hydroxy group.

### (Supplementary Note 5)

The method for detecting albumin according to Supplementary Note 4, the method being configured to utilize a phenomenon in which fluorescence emission increases due to an interaction between the terbium complex represented by Formula (1) and albumin.

### (Supplementary Note 6)

The method for detecting albumin according to Supplementary Note 4 or 5, including:
(i) obtaining a mixture of a reagent containing the terbium complex represented by Formula (1) and a sample;
(ii) irradiating the mixture with excitation light; and
(iii) detecting fluorescence emitted from the mixture.

### (Supplementary Note 7)

A terbium complex represented by Formula (1):

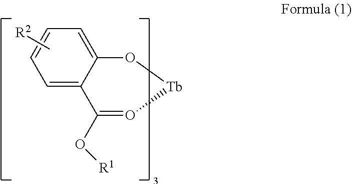

wherein R¹ represents an alkyl group having 1 to 3 carbon atoms, and R² represents a hydrogen atom, a methyl group, a methoxy group, a fluorine atom, or a hydroxy group.

### (Supplementary Note 8)

The terbium complex according to Supplementary Note 7, which is used for detecting albumin.

### (Supplementary Note 9)

A device for detecting albumin, the device including:
a capturing unit for albumin having the reagent according to any one of Supplementary Notes 1 to 3; and
a detection unit for detecting that albumin has been captured in the capturing unit.

### (Supplementary Note 10)

The device for detecting albumin according to Supplementary Note 9, wherein the detection unit detects fluorescence emission of a reactant of the terbium complex represented by Formula (1) and albumin.

### (Supplementary Note 11)

A test paper for detecting albumin, the test paper containing the reagent according to any one of Supplementary Notes 1 to 3.

### (Supplementary Note 12)

A test paper for detecting albumin, the test paper containing:
the terbium complex represented by Formula (1); and at least one selected from the group consisting of a glycol-based compound and glycerol.

### (Supplementary Note 13)

A kit for detecting albumin containing the reagent according to any one of Supplementary Notes 1 to 3.

### (Supplementary Note 14)

A system for detecting albumin which is adapted for:
obtaining in advance a reference image data showing a fluorescence emission of a reaction product between the terbium complex according to Supplementary Note 7 or 8 and albumin of a known concentration; and
comparing an image data showing a fluorescent emission of a reaction product between the terbium complex according to Supplementary Note 7 or 8 and a sample with the reference image data, to determine the concentration of albumin in the sample.

1. A reagent for detecting albumin, the reagent comprising a terbium complex represented by Formula (1): 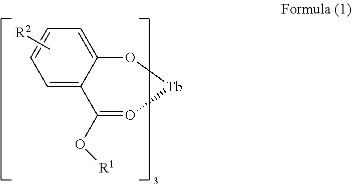
wherein R¹ represents an alkyl group having 1 to 3 carbon atoms, and R² represents a hydrogen atom, a methyl group, a methoxy group, a fluorine atom, or a hydroxy group.

2. The reagent according to claim 1, further comprising a solvent.

3. The reagent according to claim 1, further comprising at least one selected from the group consisting of a glycol-based compound and glycerol.

4. A method for detecting albumin in a sample using a terbium complex represented by Formula (1): 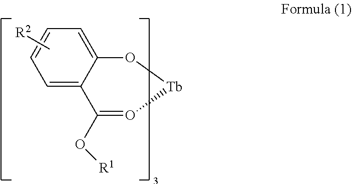
wherein R¹ represents an alkyl group having 1 to 3 carbon atoms, and R² represents a hydrogen atom, a methyl group, a methoxy group, a fluorine atom, or a hydroxy group.

5. The method for detecting albumin according to claim 4, the method being configured to utilize a phenomenon in which fluorescence emission increases due to an interaction between the terbium complex represented by Formula (1) and albumin.

6. The method for detecting albumin according to claim 4, comprising:
(i) obtaining a mixture of a reagent containing the terbium complex represented by Formula (1) and a sample;
(ii) irradiating the mixture with excitation light; and
(iii) detecting fluorescence emitted from the mixture.

7. A terbium complex represented by Formula (1): 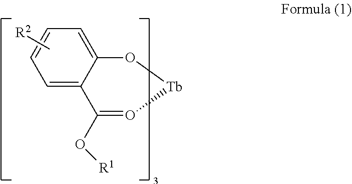
wherein R¹ represents an alkyl group having 1 to 3 carbon atoms, and R² represents a hydrogen atom, a methyl group, a methoxy group, a fluorine atom, or a hydroxy group.

8. The terbium complex according to claim 7, which is used for detecting albumin.

9. A device for detecting albumin, the device comprising:
a capturing unit for albumin having the reagent according to claim 1; and
a detection unit for detecting that albumin has been captured in the capturing unit by detecting fluorescence emission of a reactant of the terbium complex represented by Formula (1) and albumin.

10. A test paper for detecting albumin, the test paper comprising the reagent according to claim 1.

11. The reagent according to claim 2, further comprising at least one selected from the group consisting of a glycol-based compound and glycerol.

12. The test paper for detecting albumin according to claim 10, the test paper further comprising a solvent.

13. The test paper for detecting albumin according to claim 10, the test paper further comprising at least one selected from the group consisting of a glycol-based compound and glycerol.

14. A system for detecting albumin which is adapted for:
obtaining in advance a reference image data showing a fluorescence emission of a reaction product between the terbium complex according to claim 7 and albumin of a known concentration; and
comparing an image data showing a fluorescence emission of a reaction product between the terbium complex according to claim 7 and a sample with the reference image data, to determine the concentration of albumin in the sample.
